(19) United States
(12) Patent Application Publication
(10) Pub. No.: US 2020/0315482 A1
(43) Pub. Date: Oct. 8, 2020

(54) MAGNETIC MEASURING DEVICE AND HEAD-MOUNTED MAGNETIC MEASURING DEVICE

(71) Applicants: Takanobu OSAKA, Ishikawa (JP); Hironobu MIFUNE, Miyagi (JP)

(72) Inventors: Takanobu OSAKA, Ishikawa (JP); Hironobu MIFUNE, Miyagi (JP)

(73) Assignee: Ricoh Company, Ltd., Tokyo (JP)

(21) Appl. No.: 16/821,279

(22) Filed: Mar. 17, 2020

(30) Foreign Application Priority Data
Apr. 2, 2019 (JP) 2019-070737

(51) Int. Cl. A61B 5/04; G01R 33/26; G01R 33/00; A61B 5/00

(52) U.S. Cl. CPC A61B 5/04008; G01R 33/26; A61B 2562/046; A61B 5/6803; A61B 2562/0223; G01R 33/0047

(57) ABSTRACT

A magnetic measuring device includes a magnetic sensor and multiple plate parts. At least one of the plate parts holds the magnetic sensor, and the plate parts are detachably joined to each other at edges of the plate parts.

### CROSS-REFERENCE TO RELATED APPLICATION

The present application claims priority under 35 U.S.C. § 119 to Japanese Patent Application No. 2019-070737, filed on Apr. 2, 2019, the contents of which are incorporated herein by reference in their entirety.

### BACKGROUND OF THE INVENTION

### 1. Field of the Invention

An aspect of this disclosure relates to a magnetic measuring device and a head-mounted magnetic measuring device.

### 2. Description of the Related Art

A magnetic field created by a living body is called a biomagnetism. A magnetoencephalography (MEG) is a device for measuring a weak biomagnetism. The MEG measures a magnetic field (cerebral magnetic field) generated by electrical activities of nerve cells of a brain.

As an example of a magnetic sensor for magnetoencephalography, Japanese Laid-Open Patent Publication No. 2014-215151 discloses an optically-pumped atomic magnetometer that does not require a cryogenic environment.

Also, there is a known technology where a holder for holding an optically-pumped atomic magnetometer is manufactured by an additive manufacturing method to suit the shape of a face or a head (see, for example, “A new generation of magnetoencephalography: Room temperature measurements using optically-pumped magnetometers”, Neuroimage 149 (2017) 404-414, Boto E. et al.).

However, with the technology disclosed in “A new generation of magnetoencephalography: Room temperature measurements using optically-pumped magnetometers”, a holder for holding a magnetic sensor such as an optically-pumped atomic magnetometer needs to have a size that is sufficient to cover the entire head, and therefore the portability of a magnetic measuring device may be reduced. Also, Japanese Laid-Open Patent Publication No. 2014-215151 does not disclose any method for holding a magnetic sensor, and therefore cannot solve the above problem related to the portability of a magnetic measuring device capable of holding a magnetic sensor.

### SUMMARY OF THE INVENTION

According to an aspect of this disclosure, there is provided a magnetic measuring device that includes a magnetic sensor and multiple plate parts. At least one of the plate parts holds the magnetic sensor, and the plate parts are detachably joined to each other at edges of the plate parts.

### BRIEF DESCRIPTION OF THE DRAWINGS

FIG. 1 is a drawing illustrating an example of a configuration of a magnetic measuring device according to a first embodiment;

FIG. 2 is a drawing illustrating a tubular part joined to a plate part;

FIG. 3 is a perspective view of a magnetic sensor inserted into a tubular part;

FIG. 4 is a perspective view of a magnetic sensor inserted into a tubular part, where a part of the side surface of the tubular part in FIG. 3 is removed;

FIG. 5 is a drawing illustrating an example of positioning of a magnetic sensor in the longitudinal direction of a tubular part;

FIG. 6 is a drawing illustrating an example of positioning of magnetic sensors mounted on the head;

FIG. 7 is a drawing illustrating an example of a configuration of a magnetic sensor having a scale on a side surface;

FIG. 8 is an exploded perspective view illustrating an example of a configuration including a circular tubular part and a circular magnetic sensor;

FIGS. 9A and 9B are drawings used to explain the influence of directionality on the measurement sensitivity of a magnetic sensor;

FIGS. 10A and 10B are drawings illustrating examples of configurations of plate parts;

FIG. 11 is a drawing illustrating an example where plate parts are joined to each other using hinges;

FIG. 12 is a drawing illustrating an example where multiple plate parts are joined to each other;

FIG. 13 is a drawing illustrating an example where plate parts having a triangular outer shape are joined to each other;

FIGS. 14A and 14B are drawings illustrating an example of a configuration of a holder;

FIG. 15 is a drawing illustrating an example of a configuration of a magnetic measurement system including a magnetic measuring device according to the first embodiment; and

FIG. 16 is a drawing illustrating an example of a configuration of a magnetic sensor with an insulator attached to its end.

### DESCRIPTION OF THE EMBODIMENTS

In view of the above-described problem, an aspect of this disclosure makes it possible to improve the portability of a magnetic measuring device that can hold a magnetic sensor.

Embodiments of the present invention are described below with reference to the accompanying drawings. Throughout the accompanying drawings, the same reference number is assigned to the same component, and repeated descriptions of the component may be omitted.

In an embodiment described below, a head-mounted magnetic measuring device is used as an example. The head-mounted magnetic measuring device includes a magnetic measuring device including an optically-pumped atomic magnetometer and mounted on the head, and measures a magnetic field (brain magnetic field) generated by electrical activities of nerve cells of a brain. However, for brevity, in the descriptions of the embodiment, “head-mounted magnetic measuring device” is abbreviated to “magnetic measuring device”.

### First Embodiment

### Configuration of Magnetic Measuring Device of First Embodiment

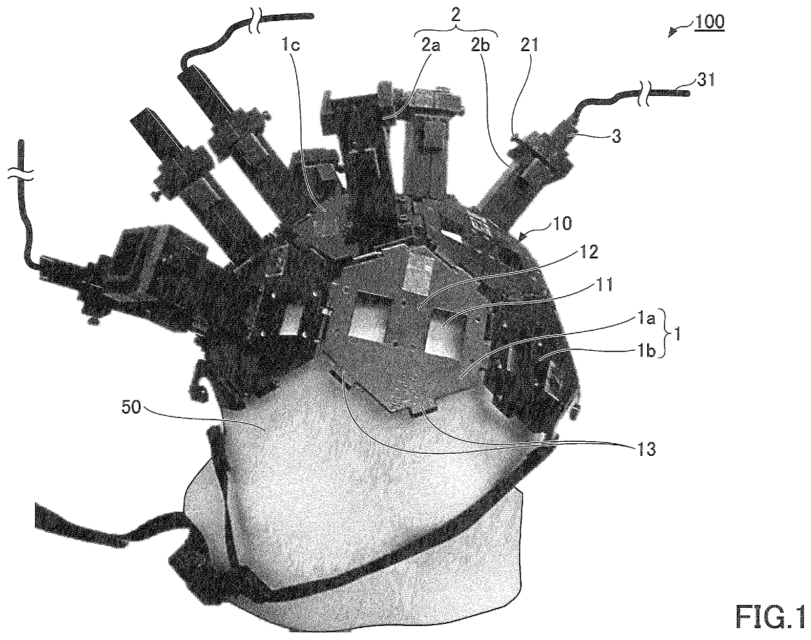

FIG. 1 is a drawing illustrating an example of a configuration of a magnetic measuring device 100 according to a first embodiment. As illustrated in FIG. 1, the magnetic measuring device 100 includes multiple plate parts 1. The multiple plate parts 1 include multiple plate parts 1a each of which includes a flat portion having a hexagonal outer shape and multiple plate parts 1b each of which includes a flat portion having a pentagonal outer shape. Hinges 13 are provided at edges of each of the plate parts 1, and the plate parts 1 are detachably joined to each other at their edges via the hinges 13.

In the descriptions below, when it is not necessary to distinguish multiple plate parts from each other, each plate part is referred to as a “plate part 1” and multiple plate parts are referred to as “plate parts 1”. When referring to a specific plate part 1, the specific plate part 1 may be indicated by a reference number with an alphabetic character such as “1a”. Such a specific plate part 1 (e.g., a plate part 1a) is also included in the plate parts 1. This method of referring to components also applies to multiple tubular parts 2 and multiple magnetic sensors 3.

The outer shape of the flat portion of the plate part 1a may be referred to as a “hexagonal shape”. However, the outer shape of the flat portion of the plate part 1a is not necessarily a complete hexagon and may include protrusions and recesses. The same applies to other shapes such as a “triangular shape”, a “pentagonal shape”, and a “circular shape”.

Referring back to FIG. 1, the entire structure formed by the joined plate parts 1 looks like one half of a soccer ball. The magnetic measuring device 100 is mounted on a head 50 such that the joined plate parts 1 cover the head 50. FIG. 1 illustrates a side view of the head 50. For brevity, the structure formed by the joined plate parts 1 is hereafter referred to as a “helmet 10”.

The plate parts 1 are attachable to and detachable from each other. That is, the plate parts 1 can be assembled, and the assembled plate parts 1 can be disassembled. In other words, the helmet 10 illustrated in FIG. 1 can be assembled and disassembled using multiple plate parts 1.

Also, as illustrated in FIG. 1, ends of the tubular parts 2 are joined to some of the plate parts 1. For example, in FIG. 1, a tubular part 2a is joined to a plate part 1c.

Each tubular part 2 is joined to a plate part 1 as described below.

Rectangular through holes are formed in each plate part 1 to pass through the plate part 1 in the thickness direction. In the example of FIG. 1, one or two rectangular through holes are formed in each plate part 1. However, the number and shape of through holes formed in each plate part 1 are not limited to this example. For example, two through holes 11 are formed in the plate part 1a. Also, four tapping holes 12 are formed around each through hole 11.

The tubular part 2 has a hollow structure, and the cross section of an internal space of the tubular part 2 in a direction orthogonal to the longitudinal direction of the tubular part 2 has a rectangular shape. An opening of the internal space at one end of the tubular part 2 has a rectangular shape, and the shape of the opening substantially matches the shape of the through hole 11 formed in the plate part 1. Here, “substantially match” may indicate, for example, a case where the shapes are the same and a case where there is a difference between the shapes that is generally recognized as a processing error. The same also applies to cases where “substantially” is used in different contexts such as “substantially aligned”.

The tubular part 2a is positioned so that the opening of the tubular part 2a is substantially aligned with a through hole formed in the plate part 1c. Then, in the aligned state, screws are inserted into four screw holes provided at an end of the tubular part 2a, and the screws are screwed into four tapping holes 12 to join the tubular part 2a to the plate part 1c. Other tubular parts 2 other than the tubular part 2a are also joined to the corresponding plate parts 1 in a similar manner.

As described above, the tubular part 2 is joined to the plate part 1 by, for example, substantially aligning the opening at one end of the tubular part 2 with the through hole in the plate part 1 and screwing the tubular part 2 and the plate part 1 together. However, the method of joining the tubular part 2 and the plate part 1 is not limited to this example. As other examples, an adhesive may be used to bond the tubular part 2 to the plate part 1, or an end of the tubular part 2 may be fitted into the through hole of the plate part 1. As still another example, the tubular part 2 may be formed in a cylindrical shape whose end is threaded, a circular tapping through hole, instead of a rectangular through hole, may be formed in the plate part 1, and the end of the tubular part 2 may be screwed into the circular tapping through hole to join the tubular part 2 to the plate part 1. Further, multiple tubular parts 2 may be joined to one plate part 1.

In the above example, the shape of the opening of the tubular part 2a substantially matches the shape of the through hole in the plate part 1c. However, the present invention is not limited to this example. Unless the space between an end of a magnetic sensor 3 inserted into the tubular part 2 and the head 50 is completely blocked by the plate part 1, the shape of the opening of the tubular part 2a is not necessarily the same as the shape of the through hole in the plate part 1c.

Referring back to FIG. 1, the magnetic sensor 3 is movably held in the internal space of the tubular part 2. More specifically, the magnetic sensor 3 has an outer shape like a rectangular column, and is inserted into the internal space of the tubular part 2b as illustrated in FIG. 1. With the magnetic sensor 3 inserted in the internal space, a screw 21 is moved from the outside of the tubular part 2b to the inside of the tubular part 2b through a tapping hole passing through a side surface of the tubular part 2b. An end of the screw 21 presses a side surface of the magnetic sensor 3 in the tubular part 2b, and the magnetic sensor 3 is thereby fixed to the tubular part 2b.

When the pressure of the screw 21 is reduced, the magnetic sensor 3 can move in the tubular part 2b in the longitudinal direction of the tubular part 2b. Thus, the magnetic sensor 3 inserted in the internal space of the tubular part 2b can be moved to a desired position in the longitudinal direction of the tubular part 2b, and can be fixed in the desired position in the longitudinal direction by pressing the magnetic sensor 3 with the screw 21. In this manner, the tubular part 2 holds the magnetic sensor 3 such that the magnetic sensor 3 is movable in the internal space.

Here, the magnetic sensor 3 is an optically-pumped atomic magnetometer that detects the intensity of a magnetic field by using spin polarization of alkali metal atoms generated by an optical pumping method. In the optical pumping method, a large difference is made between occupation numbers of atoms in two adjacent energy levels by using light.

The optically-pumped alkali metal atoms are spin-polarized. The magnetic field to be measured rotates the polarized spin and rotates the polarization plane of linearly polarized light that enters as probe light. The atomic magnetometer according to the embodiment measures the intensity of a magnetic field based on the rotation angle of the polarization plane of the probe light. The magnetic sensor 3 can output measurement data (signal) to an external device such as a personal computer (PC) via a cable 31.

The magnetic sensor 3 may be produced by using, for example, a technology described in Japanese Laid-Open Patent Publication No. 2014-215151 or a technology for detecting the intensity of light transmitted through spin-polarized alkali metal atoms (see, for example, “A new generation of magnetoencephalography: Room temperature measurements using optically-pumped magnetometers”, Neuroimage 149 (2017) 404-414, Boto E. et al.). Therefore, detailed descriptions of the magnetic sensor 3 is omitted here.

The magnetic measuring device 100 also includes a holder 4 for holding the head 50 when the helmet 10 is mounted on the head 50. The holder 4 includes a belt and holds the head 50 when the belt is fastened around a jaw included in the head 50 on which the helmet 10 is mounted. The holder 4 makes it possible to suppress the movement of the helmet 10 relative to the head 50 and to stably mount the magnetic measuring device 100 on the head 50.

In the example of FIG. 1, the tubular parts 2 are joined to some of the plate parts 1, and magnetic sensors 3 are held in some of the tubular parts 2 joined to the plate parts 1. However, the present invention is not limited to this example. The number of the tubular parts 2 and the number of the magnetic sensors 3 in relation to the number of the plate parts 1 are not limited to specific values. For example, the tubular parts 2 may be joined to all of the plate parts 1, and the magnetic sensors 3 may be provided in all of the tubular parts 2.

Also in the example of FIG. 1, the helmet 10 is shaped like one half of a soccer ball. However, the helmet 10 may be formed of a greater number of plate parts 1 so that the helmet 10 also covers areas such as the face (front side) and the back side of the head 50, and the magnetic sensors 3 are also provided in such areas.

Magnetic fields are generated in different manners depending on positions on the head 50. The magnetic measuring device 100 can measure magnetic fields in desired positions on the head 50 on which the helmet 10 is mounted by using the magnetic sensors 3 provided in desired positions on the helmet 10.

### <Details of Components>

Next, details of components of the magnetic measuring device 100 of the present embodiment are described.

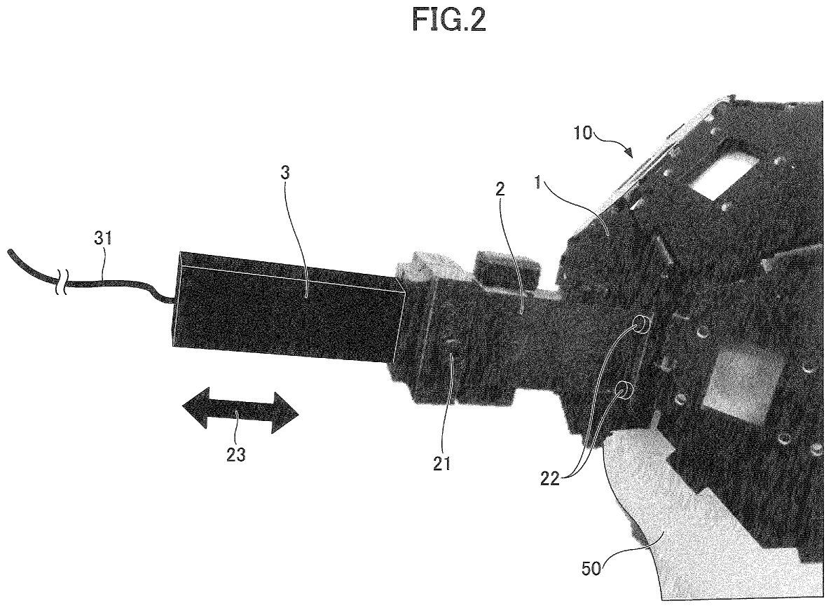

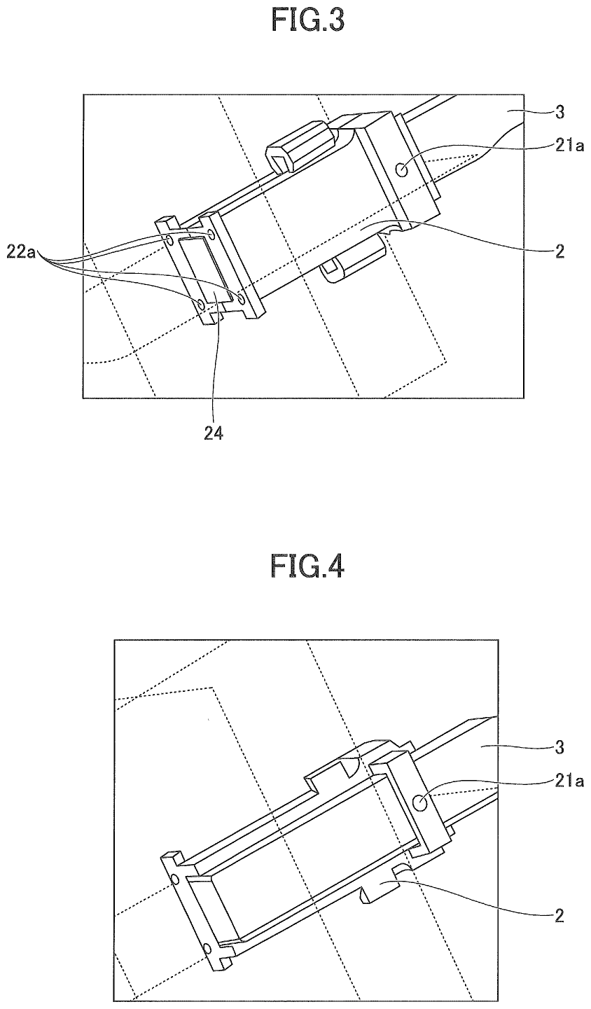

FIG. 2 is a drawing illustrating the tubular part 2 joined to the plate part 1. FIG. 2 is an enlarged view of a part of the helmet 10 mounted on the head 50. As illustrated in FIG. 2, the tubular part 2 is brought into contact with the plate part 1 such that one end of the tubular part 2 faces the flat portion of the plate part 1. Screws 22 are inserted into screw holes 22a (see FIG. 3) formed at the end of the tubular part 2, and the screws 22 are screwed into the tapping holes 12 formed around the through hole of the plate part 1 to join the tubular part 2 to the plate part 1.

The magnetic sensor 3 is inserted into the internal space of the tubular part 2 and is movable in a longitudinal direction 23 of the tubular part 2 indicated by a thick arrow. After being moved to a desired position in the longitudinal direction 23, the magnetic sensor 3 is fixed to the tubular part 2 by a screw 21 inserted into a tapping hole passing through a side surface of the tubular part 2, and is thereby held in the tubular part 2.

FIG. 3 is a perspective view of the magnetic sensor 3 inserted into the tubular part 2. As illustrated in FIG. 3, the magnetic sensor 3 is inserted into the internal space of the tubular part 2 from one end of the tubular part 2 on the right side in FIG. 3 and passes through the tubular part 2 to the other end of the tubular part 2 on the left side in FIG. 3. In FIG. 3, an end of the magnetic sensor 3 passing through the internal space of the tubular part 2 is visible in an opening 24 at the other end of the tubular part 2. The magnetic sensor 3 is fixed to the tubular part 2 by the screw 21 (see FIG. 2) inserted into a tapping hole 21a that passes through the side surface of the tubular part 2, and is thereby held in the tubular part 2.

The tubular part 2 is positioned on the plate part 1 such that the opening 24 of the tubular part 2 is substantially aligned with the through hole 11 (see FIG. 1) of the plate part 1. Then, the screws 22 (see FIG. 2) are inserted, from right to left in FIG. 3, into the four screw holes 22a formed at the end of the tubular part 2, and the screws 22 are screwed into the four tapping holes 12 (see FIG. 1) in the plate part 1 to join the tubular part 2 to the plate part 1. As a result, the end of the magnetic sensor 3 passing through the internal space of the tubular part 2 passes through the through hole 11 of the plate part 1 to face the head 50 and is positioned close to or brought into contact with the head 50.

FIG. 4 is a perspective view of the magnetic sensor 3 inserted into the tubular part 2, where a part of the side surface of the tubular part 2 in FIG. 3 is removed. As illustrated in FIG. 4, the magnetic sensor 3 is inserted into the internal space of the tubular part 2, is fixed to the tubular part 2 by the screw 21 (see FIG. 2) passing through the tapping hole 21a, and is thereby held in the tubular part 2.

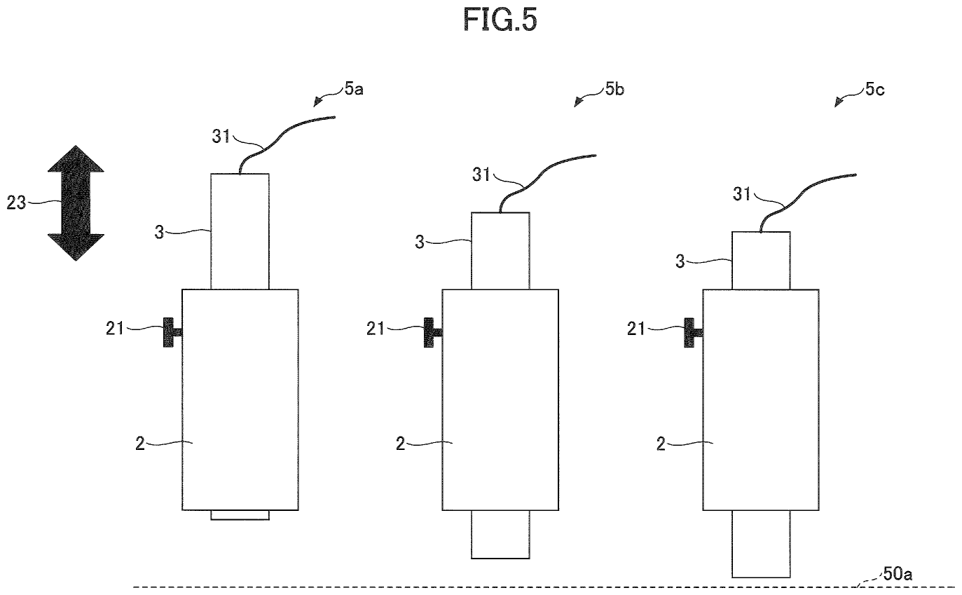

FIG. 5 is a drawing illustrating an example of positioning of the magnetic sensor 3 in the longitudinal direction 23 of the tubular part 2. FIG. 5 illustrates three states 5a to 5c where the magnetic sensor 3 is inserted in the internal space of the tubular part 2 and moved in the longitudinal direction 23. In FIG. 5, a dotted line indicates a surface 50a of the head 50.

In the state 5a, an end of the magnetic sensor 3 (which faces the surface 50a) is in a position that is farthest from the surface 50a among the states 5a to 5c. In the state 5b, the magnetic sensor 3 is moved in the longitudinal direction 23 so that the end of the magnetic sensor 3 is positioned closer to the surface 50a than in the state 5a. In the state 5c, the magnetic sensor 3 is moved further in the longitudinal direction 23 from the position in the state 5b. By moving the magnetic sensor 3 in the longitudinal direction 23, the position of the end of the magnetic sensor 3 relative to the surface 50a of the head 50 can be changed as in the states 5a to 5c. When the end of the magnetic sensor 3 is placed in a desired position relative to the surface 50a of the head 50, the magnetic sensor 3 is fixed to and held in the tubular part 2 with the screw 21.

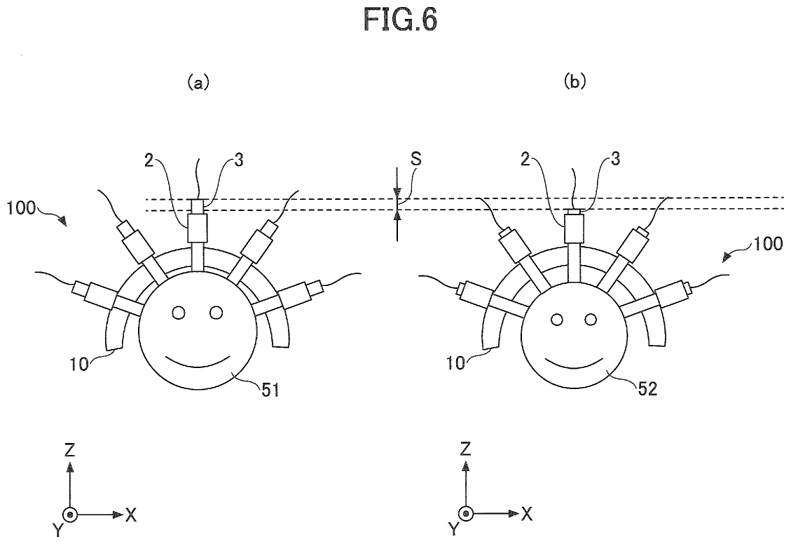

FIG. 6 is a drawing illustrating an example of positioning of the magnetic sensors 3 mounted on the head 50. FIG. 6 (a) illustrates a state where the magnetic sensors 3 are mounted on a large head 51, and FIG. 6 (b) illustrates a state where the magnetic sensors 3 are mounted on a small head 52. For example, the head 51 is a head of an adult, and the head 52 is a head of a child that is smaller than the head of the adult.

The position and the shape of the helmet 10 are the same in FIG. 6 (a) and FIG. 6 (b). However, because the head 52 is smaller than the head 51, the distance between the helmet 10 and the head 52 in FIG. 6 (b) is greater than the distance between the helmet 10 and the head 51 in FIG. 6 (a). The sensitivity of the magnetic sensor 3 in measuring the magnetic field varies depending on the position (distance) of the end of the magnetic sensor 3 relative to the head. Accordingly, the measurement sensitivity of the magnetic measuring device 100 in the state of FIG. 6 (a) in measuring the magnetic field of the head 51 is different from that in measuring the magnetic field of the head 52.

For this reason, as illustrated in FIG. 6 (b), the magnetic sensor 3 is moved in the longitudinal direction of the tubular part 2 by a movement amount S toward the head 52 to adjust the position of the end of the magnetic sensor 3 relative to the head 52. This configuration makes it possible to place the end of the magnetic sensor 3 in a predetermined position relative to the head 52 and to measure the magnetic field with a predetermined measurement sensitivity. Here, because the helmet 10 has a shape that curves along the head, as illustrated in FIG. 6, the longitudinal direction of the tubular part 2 varies depending on the position of the magnetic sensor 3 on the helmet 10. Therefore, the positions of the ends of the different magnetic sensors 3 are adjusted in different directions. Also, the magnetic sensors 3 may be moved by different amounts according to the shape of the head so that the ends of the magnetic sensors 3 are placed in a predetermined position relative to the head.

The positions of the respective magnetic sensors 3 arranged on the helmet 10 are determined based on the design values of the helmet 10, the tubular parts 2, and the magnetic sensors 3. Therefore, the positions of the ends of the respective magnetic sensors 3 on the helmet 10 are preferably managed using the three-dimensional XYZ coordinate system illustrated in FIG. 6. This method makes it possible to quantitatively manage the positions of the ends of the magnetic sensors 3 relative to the head and to measure magnetic fields at a predetermined measurement sensitivity with the magnetic sensors 3 regardless of the size and the shape of the head.

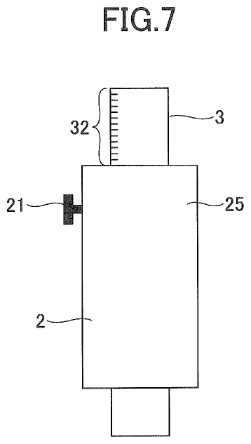

Here, the movement amount S of the magnetic sensor 3 can be quantitatively managed by providing a scale on a side surface of the magnetic sensor 3. FIG. 7 is a drawing illustrating an example of a configuration of the magnetic sensor 3 having a scale on a side surface. As illustrated in FIG. 7, a scale 32 is provided on a side surface of the magnetic sensor 3. The position or the movement amount of the magnetic sensor 3 relative to an end face 25 of the tubular part 2 can be quantitatively determined by visually observing the position of the end face 25 on the scale 32.

In addition to or instead of visually observing the scale 32, an image of the scale 32 and the end face 25 may be captured with a camera, and the captured image may be processed to obtain the position of the end face 25 on the scale 32. Compared with visual observation, using image capturing by a camera and image processing make it possible more accurately detect the position or the movement amount of the magnetic sensor 3 relative to the end face 25.

However, the methods for detecting the position or the movement amount of the magnetic sensor 3 relative to the end face 25 are not limited to those described above. As another example, the position or the movement amount of the magnetic sensor 3 may be detected by reading a linear scale provided on the magnetic sensor 3 with an encoder provided on the tubular part 2. Using a linear scale makes it possible to more accurately detect the position or the movement amount of the magnetic sensor 3. On the other hand, compared with a case where a linear scale is used, using image capturing by a camera and image processing makes it possible to detect the position or the movement amount of the magnetic sensor 3 relative to the end face 25 at lower cost.

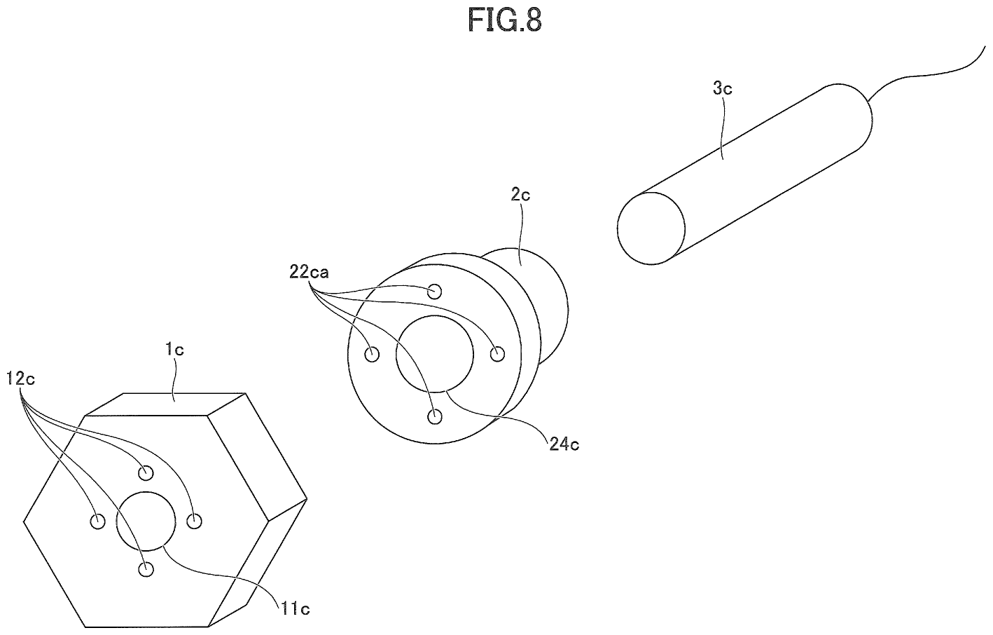

FIG. 8 is an exploded perspective view illustrating an example of a configuration including a circular tubular part and a circular magnetic sensor. As illustrated in FIG. 8, a magnetic sensor 3c has a cylindrical shape, and a tubular part 2c has a cylindrical shape. Also, the cross section of the internal space of the tubular part 2c and an opening 24c of the internal space have circular shapes.

The magnetic sensor 3c is movably held in the internal space of the tubular part 2c. More specifically, with the magnetic sensor 3c inserted in the internal space of the tubular part 2c, a screw is moved from the outside of the tubular part 2c to the inside of the tubular part 2c through a tapping hole passing through a side surface of the tubular part 2c. An end of the screw presses the side surface of the magnetic sensor 3c in the tubular part 2c to fix the magnetic sensor 3c to the tubular part 2c. When the screw is loosened, the magnetic sensor 3c can be moved in the internal space of the tubular part 2c in the longitudinal direction of the tubular part 2c. After the magnetic sensor 3c is moved to a desired position, the screw is tightened to fix the magnetic sensor 3c.

While one end of the tubular part 2c is in contact with the plate part 1c, screws are inserted into through holes 22ca formed at the end of the tubular part 2c and screwed into tapping holes 12c to join the tubular part 2c to the plate part 1c. The end of the magnetic sensor 3c faces the head 50 via a through hole 11 of the plate part 1c. With the end of the magnetic sensor 3c facing the head 50, the magnetic sensor 3c can measure the magnetic field of the head 50.

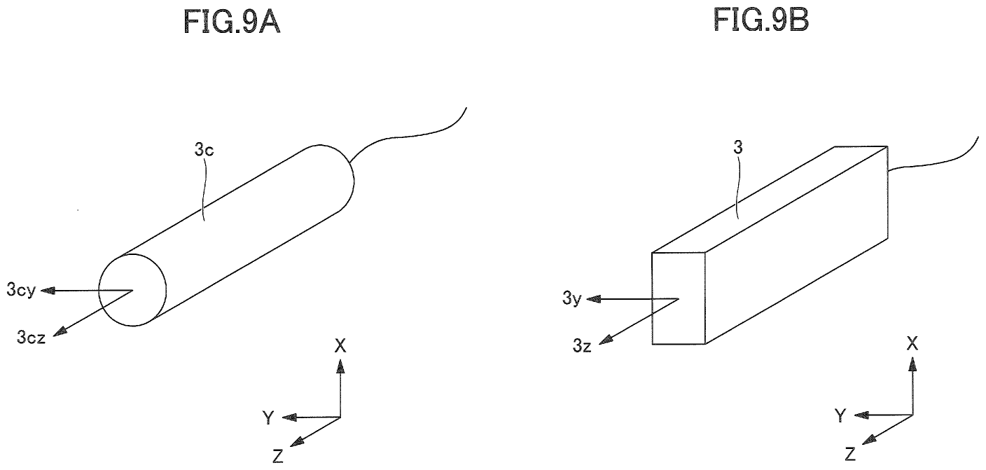

The measurement sensitivity of the magnetic sensor 3, which is an optically-pumped atomic magnetometer, has directionality and depending on the method of holding the magnetic sensor 3, this directionality may influence the measurement of the magnetic field. FIGS. 9A and 9B are drawings used to explain the influence of directionality on the measurement sensitivity of the magnetic sensor 3. FIG. 9A illustrates a case where the magnetic sensor has a cylindrical outer shape, and FIG. 9B illustrates a case where the magnetic sensor has an outer shape like a rectangular column.

In FIG. 9A, the magnetic sensor 3c having a cylindrical outer shape can measure a magnetic field in the direction of an arrow 3cy and a magnetic field in the direction of an arrow 3cz. Direction data of magnetic fields can be accurately obtained based on measurement signals of the magnetic sensor 3c by adjusting the magnetic sensor 3c in advance such that the direction of the arrow 3cy corresponds to the Y-axis direction and the direction of the arrow 3cz corresponds to the Z-axis direction.

In the case of FIG. 9A, because the magnetic sensor 3c has a cylindrical outer shape whose cylindrical axis corresponds to the Z-axis direction, the magnetic sensor 3c may rotate around the Z axis due to, for example, vibration or a sudden impact applied to the magnetic measuring device 100. Also, when the magnetic sensor 3c rotates around the Z axis, it is difficult to detect the rotation of the magnetic sensor 3c. If the magnetic sensor 3c rotates around the Z-axis, the direction of the arrow 3cy deviates from the Y-axis direction, and the direction data of magnetic fields may not be accurately obtained based on measurement signals of the magnetic sensor 3c.

In the case of FIG. 9B, the magnetic sensor 3 has an outer shape like a rectangular column, and the magnetic sensor 3 can measure a magnetic field in the direction of an arrow 3y and a magnetic field in the direction of an arrow 3z. Direction data of magnetic fields can be accurately obtained based on measurement signals of the magnetic sensor 3 by adjusting the magnetic sensor 3 in advance such that the direction of the arrow 3y corresponds to the Y-axis direction and the direction of the arrow 3z corresponds to the Z-axis direction.

Because the magnetic sensor 3 has an outer shape like a rectangular column, even when vibration or a sudden impact is applied to the magnetic measuring device 100, the rotation of the magnetic sensor 3 around the Z axis can be suppressed compared with the cylindrical magnetic sensor 3c. Also, even if the magnetic sensor 3 rotates around the Z axis, because the magnetic sensor 3 tilts as a result of the rotation, the rotation of the magnetic sensor 3 can be easily detected, and the magnetic sensor 3 can be adjusted again to cancel the rotation. Thus, the direction data of magnetic fields can be accurately obtained based on measurement signals of the magnetic sensor 3.

Thus, the robustness of the magnetic measuring device 100 against, for example, vibration or a sudden impact can be improved by using the magnetic sensor 3 with an outer shape like a rectangular column (or a rectangular shape) and by holding the magnetic sensor 3 with the tubular part 2 having an internal space whose cross section orthogonal to the longitudinal direction of the tubular part 2 has a rectangular shape.

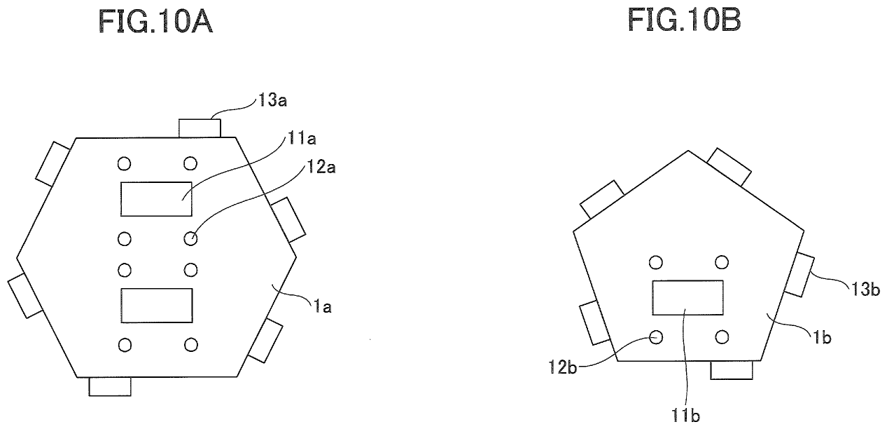

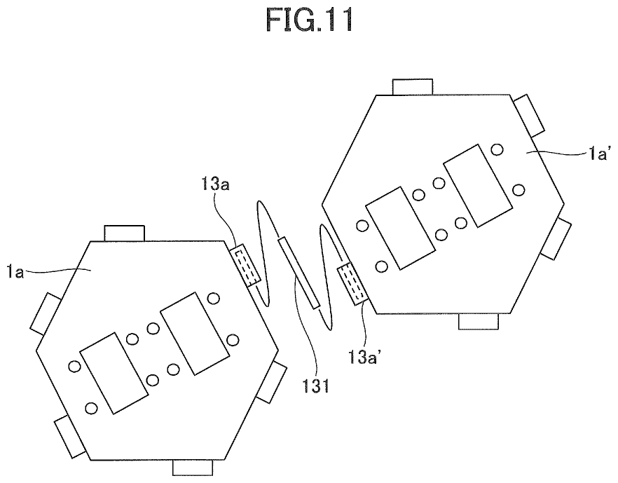

Next, configurations of the plate parts 1 are described in detail. FIGS. 10A and 10B are drawings illustrating examples of configurations of the plate parts 1. FIG. 10A illustrates a plate part 1a having a hexagonal outer shape, and FIG. 10B illustrates a plate part 1b having a pentagonal outer shape. FIGS. 10A and 10B illustrate states where the tubular parts 2 are not joined to the plate parts 1a and 1b.

In FIG. 10A, the plate part 1a includes two through holes 11a, and four tapping holes 12a formed around each of the two through holes 11a. As described above, the tubular part 2 is positioned so that the opening of the tubular part 2 is substantially aligned with the through hole 11a, and the tubular part 2 is joined to the plate part 1a by screwing screws into the four tapping holes 12a. Hinges 13a are provided on the outer periphery of the plate part 1a. One hinge 13a is provided on each side of the hexagonal shape, and a total of six hinges 13a are provided.

In FIG. 10B, the plate part 1b includes one through hole 11b and four tapping holes 12b formed around the through hole 11b. As described above, the tubular part 2 is positioned so that the opening of the tubular part 2 is substantially aligned with the through hole 11b, and the tubular part 2 is joined to the plate part 1b by screwing screws into the four tapping holes 12b. Also, hinges 13b are provided on the outer periphery of the plate part 1b. One hinge 13b is provided on each side of the pentagonal shape, and a total of five hinges 13b are provided.

In the examples of FIGS. 10A and 10B, the plate part 1a includes two through holes 11a, and the plate part 1b includes one through hole 11b. However, the present invention is not limited to these examples. For example, the plate part 1a may have one through hole 11a, the plate part 1b may have two through holes 11b, or both of the plate parts 1a and 1b may have one or two through holes.

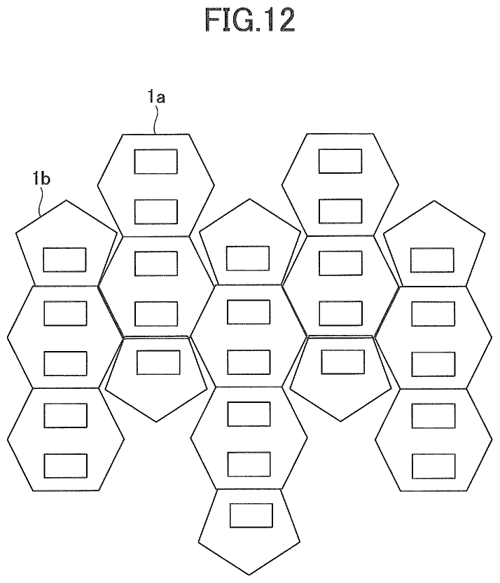

FIG. 11 is a drawing illustrating an example where the plate parts 1 are joined to each other using the hinges 13. In the example of FIG. 11, two plate parts 1a and 1a′ having a hexagonal shape are joined to each other.

The hinge 13a provided on the outer periphery of the plate part 1a includes a hole (or an internal space) into which the pin 131 is to be inserted. Similarly, a hinge 13a′ provided on the outer periphery of the plate part 1a′ also includes a hole (or an internal space) into which the pin 131 is to be inserted.

The hinge 13a and the hinge 13a′ are positioned symmetrically with respect to the centers of the sides of the plate parts 1a and 1a′. As illustrated in FIG. 11, the same pin 131 is inserted into both of the hinges 13a and 13a′ to join the plate part 1a and the plate part 1a′ together.

Using the hinges 13 to join the plate parts 1 makes it possible to move the joined plate parts 1 to match the three-dimensional shape of the head 50. This in turn makes it possible to change the shape of the helmet 10 to match the three-dimensional shape of the head 50, and thereby makes it possible to more stably mount the magnetic measuring device 100 on the head 50. Also, using the hinges 13 makes it possible to easily assemble and disassemble the plate parts 1. In other words, using the hinges 13 makes it possible to easily attach and detach the plate parts 1 to and from each other.

In the example of FIG. 11, hexagonal plate parts 1a are joined to each other. However, a hexagonal plate part 1a may be joined to a pentagonal plate part 1b, and pentagonal plate parts 1b may be joined to each other. In any case, the plate parts 1 may be joined to each other using the hinges 13 as in the above-described example.

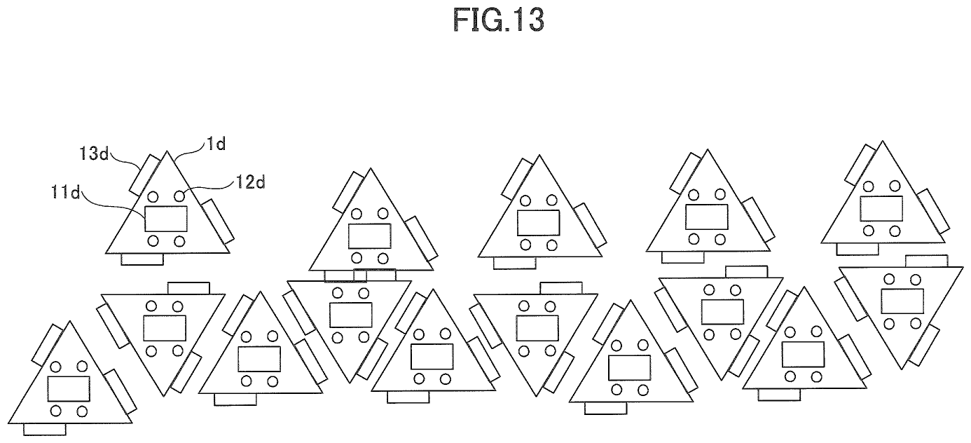

FIG. 12 is a drawing illustrating an example where multiple plate parts are joined to each other. FIG. 12 illustrates an example where plate parts 1a having a regular hexagonal outer shape and plate parts 1b having a regular pentagonal outer shape are combined and joined to each other. More specifically, FIG. 12 illustrates an example where ten plate parts 1a and six plate parts 1b are combined and joined to each other. With this combination of the plate parts 1a and 1b, the helmet 10 shaped like one half of a soccer ball as in FIG. 1 is formed. In FIG. 12, hinges and tapping holes formed in each of the plate parts 1a and 1b are omitted.

The configuration of the helmet 10 shaped like one half of a soccer ball makes it possible to form the helmet 10 while reducing unnecessary space between the plate parts 1.

In the above-described example, the plate parts 1a having a regular hexagonal outer shape and the plate parts 1b having a regular pentagonal outer shape are combined and joined to each other. However, the present invention is not limited to this example. For example, plate parts having other outer shapes such as a triangular shape, a rectangular shape, and a circular shape may also be used.

FIG. 13 is a drawing illustrating an example where plate parts having a triangular outer shape are joined to each other. As illustrated in FIG. 13, plate parts 1d have a triangular outer shape. Similarly to the plate part 1a described above, each plate part 1d includes a through hole 11d and four tapping holes 12d formed around the through hole 11d. Also, a hinge 13d is provided on each side of the periphery of the plate part 1d. Functions of the through hole 11d, the tapping holes 12d, and the hinges 13d are substantially the same as the corresponding components of the plate part 1a, and therefore descriptions of these components are omitted here.

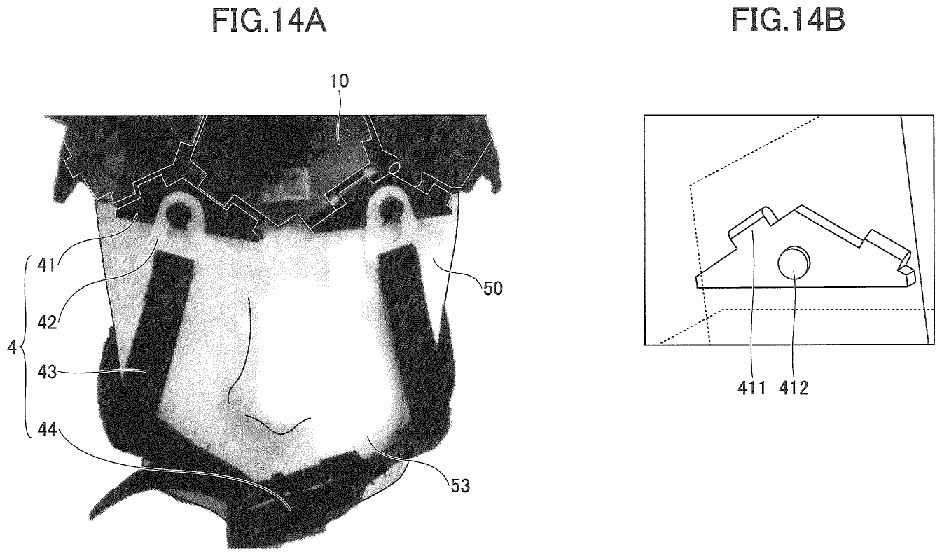

Next, a configuration of the holder 4 of the magnetic measuring device 100 is described. FIGS. 14A and 14B are drawings illustrating an example of a configuration of the holder 4. FIG. 14A illustrates a state where the holder 4 attached to the helmet 10 holds the head 50, and FIG. 14B illustrates an example of a configuration of a coupling plate part 41 included in the holder 4.

As illustrated in FIG. 14A, the holder 4 includes coupling plate parts 41, hooks 42, belts 43, and a buckle 44. The holder 4 includes two coupling plate parts 41, two hooks 42, and two belts 43.

Each of the two coupling plate parts 41 is used to connect the holder 4 to the plate parts 1 of the helmet 10. In the example of FIG. 14, the coupling plate part 41 has an outer shape like an isosceles triangle, and a hinge 411 is provided on each of the two equal sides. The hinge 411 includes a hole (or an internal space) into which the pin 131 (see FIG. 11) is to be inserted. The coupling plate part 41 is joined to the plate part 1 by inserting the pin 131 into both of the hole in the hinge 411 and the hole in the hinge 13 of the plate part 1. As illustrated in FIG. 14A, the two coupling plate parts 41 are joined to the helmet 10 at two positions.

Also, a protrusion 412 is formed on a flat portion of each of the coupling plate parts 41. The hook 42 is hooked on the protrusion 412. The hooks 42 attached to ends of the two belts 43 are hooked on the protrusions 412 of the coupling plate parts 41, and the two belts 43 are thereby connected to the coupling plate parts 41. The buckle 44 is attached to the other ends of the two belts 43.

The two belts 43 are connected via the coupling plate parts 41 to the helmet 10 mounted on the head 50, and the buckle 44 is fastened to connect the two belts 43 to each other such that the connected belts 43 are wrapped around a jaw 53 of the head 50. The lengths of the belts 43 are adjusted in advance so that the belts 43 wrapped around the jaw 53 do not move when the buckle 44 is fastened. In this manner, the holder 4 holds the head 50.

Here, the magnetic measuring device 100 is mounted on the head 50 such that the helmet 10 covers the head 50. The magnetic sensors 3 included in the magnetic measuring device 100 are connected to ends of cables for transmitting measurement data and driving the magnetic sensors 3, and the other ends of the cables are connected to a controller such as a PC. Therefore, when the head 50 moves or a person or an object touches the cables, the magnetic measuring device 100 may be misaligned with the head 50. If the magnetic measuring device 100 is misaligned with the head 50 during measurement of magnetic fields, measurement errors of the magnetic fields may occur.

With the present embodiment, however, because the holder 4 holds the head 50, misalignment of the magnetic measuring device 100 with the head 50 can be prevented even if the head 50 moves or a person or an object touches the cables during the measurement of magnetic fields.

In the above example, the coupling plate part 41 has an outer shape like an isosceles triangle. However, the coupling plate part 41 may have any other outer shape. Nevertheless, the coupling plate part 41 preferably has an outer shape like an isosceles triangle because an isosceles triangular shape fits in a recess formed in a rugged edge of the helmet 10 and can flatten (or smooth) the edge of the helmet 10.

The plate parts 1, the tubular parts 2, and the holder 4 described above are preferably formed of a non-magnetic material. Using a non-magnetic material makes it possible to reduce measurement noise of the magnetic sensors 3 and makes it possible to more accurately measure magnetic fields of the head 50, which is a measurement target.

For example, the plate part 1 and the tubular part 2 are produced using a three-dimensional (3D) printer. There are various types of three-dimensional printers employing, for example, a powder additive manufacturing method, a hot melt laminating method, and an optical molding method. Any type of three-dimensional printer may be used for this purpose. Among these methods, the powder additive manufacturing method is particularly preferable because the powder additive manufacturing method can use various materials and can form a complicated three-dimensional shape.

If a helmet to be mounted on the head 50 is produced monolithically using a three-dimensional printer, a large three-dimensional printer capable of producing a helmet as large as the head 50 is necessary, and the production costs may increase.

In contrast, in the present embodiment, the helmet 10 is assembled by joining multiple plate parts 1, and the magnetic measuring device 100 is produced by joining the tubular parts 2 to the helmet 10. Therefore, only small components such as the plate parts 1 and the tubular parts 2 need to be produced with a three-dimensional printer. Accordingly, the present embodiment makes it possible to produce components with a small three-dimensional printer and to reduce the production costs.

Components of the holder 4 except for the belts 43 can also be produced with a three-dimensional printer. The belts 43 are preferably formed of, for example, a flexible cloth or a resin to be able to stably hold the head 50.

### <Effects>

As described above, in the present embodiment, the helmet 10 used to mount the magnetic measuring device 100 on the head 50 is formed by detachably joining the edges of multiple plate parts 1 for holding the magnetic sensors 3. This configuration makes it possible to assemble the magnetic measuring device 100 by joining multiple plate parts 1 together, and to disassemble the magnetic measuring device 100 into the plate parts 1 by disjoining the plate parts 1. Compared with a case where the helmet 10 is monolithically formed, the configuration of the present embodiment where the helmet 10 can be disassembled into small and portable plate parts 1 makes it possible to improve the portability of the magnetic measuring device 100.

Also, the magnetic measuring device 100 of the present embodiment includes the tubular parts 2. One end of each tubular part 2 is joined to the plate part 1, and the magnetic sensor 3 is movably held in an internal space of the tubular part 2. Thus, the plate part 1 holds the magnetic sensor 3 via the tubular part 2. This configuration makes it possible to adjust the position (distance) of the magnetic sensor 3 relative to the head 50 on which the magnetic measuring device 100 is mounted, and thereby makes it possible to measure magnetic fields of a measurement target such as the head 50 at a predetermined sensitivity without being influenced by, for example, differences in size and shape of the head 50.

Also in the present embodiment, the plate parts 1 have a polygonal shape. This makes it possible to form the helmet 10 while reducing unnecessary space between the plate parts 1. Further, the helmet 10 may also be formed by combining hexagonal plate parts 1a and pentagonal plate parts 1b. This configuration makes it possible to further reduce unnecessary space between the plate parts 1 and to form the helmet 10 that is a part of a sphere.

In the present embodiment, the hinges 13 are used to join the plate parts 1 to each other. With this configuration, the joined plate parts 1 can be moved according to the three-dimensional shape of the head 50. This in turn makes it possible to change the shape of the helmet 10 to match the three-dimensional shape of the head 50, and thereby makes it possible to more stably mount the magnetic measuring device 100 on the head 50. Further, using the hinges 13 to join the plate parts 1 makes it easier to join and separate the plate parts 1.

In the present embodiment, at least one of the plate part 1 and the tubular part 2 is formed of a non-magnetic material to reduce measurement noise of the magnetic sensor 3 and to accurately measure the magnetic fields of the head 50, which is a measurement target.

Also in the present embodiment, the magnetic sensor 3 has a rectangular outer shape, and the magnetic sensor 3 is held by the tubular part 2 having an internal space whose cross section orthogonal to the longitudinal direction of the tubular part 2 has a rectangular shape. This configuration makes it possible to prevent the rotation of the magnetic sensor 3 even when vibration or a sudden impact is applied to the magnetic measuring device 100. Also, even if the magnetic sensor 3 rotates, the rotation of the magnetic sensor 3 can be easily detected, and the magnetic sensor 3 can be adjusted again to cancel the rotation. Thus, the present embodiment makes it possible to improve the robustness of the magnetic measuring device 100 against, for example, vibration or a sudden impact.

In the present embodiment, the magnetic measuring device 100 includes the holder 4 for holding the head 50. With the holder 4 for holding the head 50, the magnetic measuring device 100 can be stably mounted on the head 50, and the magnetic sensor 3 can be stably held relative to the head 50.

### <Example of Application to Magnetic Measurement System>

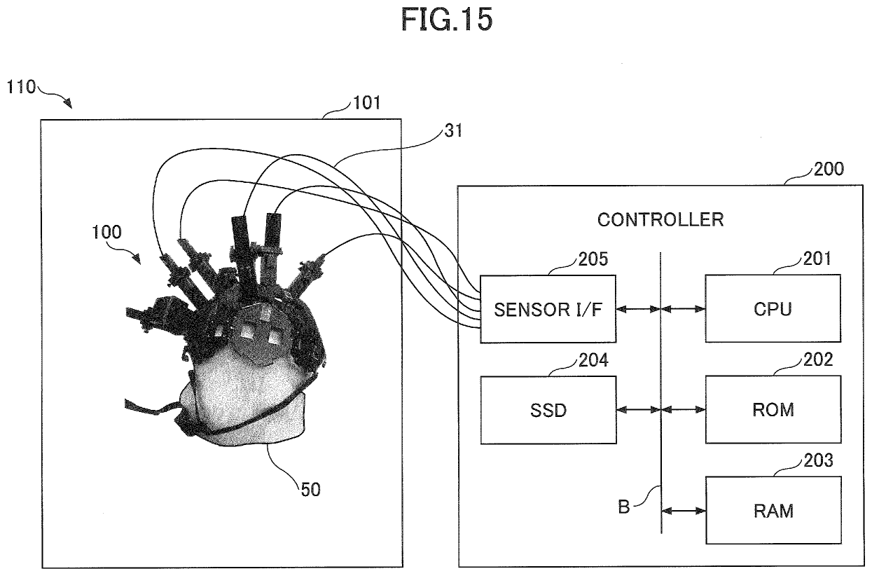

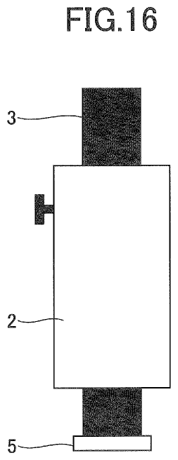

As a variation of the magnetic measuring device 100 of the present embodiment, an application of the magnetic measuring device 100 to a magnetic measurement system is described. FIG. 15 is a drawing illustrating an example of a configuration of a magnetic measuring system 110 including the magnetic measuring device 100.

As illustrated in FIG. 15, the magnetic measuring system 110 includes the magnetic measuring device 100 and a controller 200. The magnetic measuring device 100 is placed in a magnetic shield room 101 and is electrically connected via cables 31 to the controller 200 such as a PC disposed outside of the magnetic shield room 101.

Placing the magnetic measuring device 100 in the magnetic shield room 101 makes it possible to block geomagnetism that causes measurement noise, and thereby makes it possible to more accurately measure magnetic fields. As illustrated in FIG. 15, when measuring magnetic fields of a brain, a test subject enters the magnetic shield room 101, and the magnetic measuring device 100 is mounted on the head 50 of the test subject.

As illustrated in FIG. 15, the controller 200 includes a central processing unit (CPU) 201, a read-only memory (ROM) 202, a random access memory (RAM) 203, a solid state drive (SSD) 204, and a sensor Interface (I/F) 205. These components are electrically connected to each other via a system bus B.

The CPU 201 reads programs and data from a storage device such as the ROM 202 or the SSD 204, loads the programs and data onto the RAM 203, and executes the programs to control the entire controller 200 and implement functions of the controller 200. A part or all of the functions of the CPU 201 may also be implemented by an electronic circuit such as an application specific integrated circuit (ASIC) or a field-programmable gate array (FPGA).

The ROM 202 is a nonvolatile semiconductor memory (storage device) that can retain programs and data even when the power is turned off. The ROM 202 stores programs and data such as a basic input/output system (BIOS) executed when the controller 200 is started and operating system (OA) settings. The RAM 203 is a volatile semiconductor memory (storage device) that temporarily stores programs and data.

The SSD 204 is a non-volatile memory that stores programs for implementing processes performed by the controller 200 and various types of data. The SSD 204 may be replaced with, for example, a hard disk drive (HDD).

The operation of the magnetic sensor 3 is controlled by a drive signal from the controller 200, and measurement data of the magnetic sensor 3 is transmitted from the magnetic sensor 3 to the controller 200. In FIG. 15, for brevity, each cable 31 is illustrated as one cable. However, it is assumed that each cable 31 includes a drive signal cable and a measurement data transmission cable separately.

Measurement data of multiple magnetic sensors 3 is transmitted to the controller 200, and the CPU 201 executes programs to perform various types of data processing and data analysis for measurement of magnetic fields.

### Second Embodiment

Next, a magnetic measuring device 100a according to a second embodiment is described. Descriptions of components that are the same as those described in the first embodiment are omitted here.

In an optically-pumped atomic magnetometer, which is an example of a magnetic sensor, it is necessary to heat a glass cell (gas cell) containing an alkali metal gas to vaporize the alkali metal gas. In the case of potassium, the temperature in the glass cell becomes about 100 degrees Celsius. In terms of safety, it is not preferable that the heat of the magnetic sensor is transferred to a measurement target such as a head on which the magnetic measuring device is mounted.

For this reason, the magnetic measuring device 100a of the second embodiment includes an insulator 5 provided at an end of the magnetic sensor 3 facing the head 50. FIG. 16 is a drawing illustrating an example of a configuration of a magnetic sensor 3 at an end of which the insulator 5 is provided.

Examples of the insulator 5 may include a fiber insulator such as glass wool or rock wool, a foam insulator such as urethane foam or phenol foam, and aerogel. The insulator 5 may be attached as a component to the end of the magnetic sensor 3, or the insulator 5 may be implemented by coating the end of the magnetic sensor 3 with an insulating material.

Providing the insulator 5 makes it possible to prevent the heat of the magnetic sensor 3 from being transferred to the head 50, and thereby makes it possible to more safely and reliably measure magnetic fields.

Other effects of the magnetic measuring device 100a of the second embodiment are substantially the same as those of the magnetic measuring device 100 described in the first embodiment.

A magnetic measuring device and a head-mounted magnetic measuring device according to the embodiments of the present invention are described above. However, the present invention is not limited to the specifically disclosed embodiments, and variations and modifications may be made without departing from the scope of the present invention.

What is claimed is:

1. A magnetic measuring device, comprising:
a magnetic sensor; and
multiple plate parts, at least one of the plate parts holding the magnetic sensor,
wherein the plate parts are detachably joined to each other at edges of the plate parts.

2. The magnetic measuring device as claimed in claim 1, further comprising:
a tubular part that includes an internal space where the magnetic sensor is movably held, an end of the tubular part being joined to the at least one of the plate parts,
wherein the at least one of the plate parts holds the magnetic sensor via the tubular part.

3. The magnetic measuring device as claimed in claim 2, wherein
each plate part of the plate parts includes a through hole that passes through the plate part in a thickness direction of the plate part; and
a shape of an opening at the end of the tubular part matches a shape of the through hole.

4. The magnetic measuring device as claimed in claim 1, wherein each of the plate parts has a polygonal outer shape.

5. The magnetic measuring device as claimed in claim 1, wherein
some of the plate parts have a pentagonal outer shape; and
some of the plate parts have a hexagonal outer shape.

6. The magnetic measuring device as claimed in claim 2, wherein the plate parts, the tubular part, or both of the plate parts and the tubular part are formed of a non-magnetic material.

7. The magnetic measuring device as claimed in claim 1, wherein the plate parts are detachably joined to each other via hinges.

8. The magnetic measuring device as claimed in claim 7, wherein the hinges are formed of a non-magnetic material.

9. The magnetic measuring device as claimed in claim 2, wherein a cross section of the internal space in a direction orthogonal to a longitudinal direction of the tubular part has a rectangular shape.

10. The magnetic measuring device as claimed in claim 1, wherein the magnetic sensor is an optically-pumped atomic magnetometer.

11. The magnetic measuring device as claimed in claim 1, wherein the magnetic sensor includes an insulator disposed to face a measurement target.

12. A head-mounted magnetic measuring device, comprising:
a magnetic sensor; and
multiple plate parts configured to cover a head, at least one of the plate parts holding the magnetic sensor,
wherein the plate parts are detachably joined to each other at edges of the plate parts.

13. The head-mounted magnetic measuring device as claimed in claim 12, further comprising:
a holder that holds the head.
